(19) United States
(12) Patent Application Publication
(10) Pub. No.: US 2024/0067919 A1
(43) Pub. Date: Feb. 29, 2024

(54) DENSITY ADJUSTING APPARATUS AND DENSITY-ADJUSTED LIQUID PRODUCING METHOD

(71) Applicants: CANON MEDICAL SYSTEMS CORPORATION, Otawara-shi (JP); Canon Kabushiki Kaisha, Tokyo (JP)

(72) Inventor: Kenta KAMINO, Utsunomiya (JP)

(73) Assignees: CANON MEDICAL SYSTEMS CORPORATION, Otawara-shi (JP); Canon Kabushiki Kaisha, Tokyo (JP)

(21) Appl. No.: 18/453,384

(22) Filed: Aug. 22, 2023

(30) Foreign Application Priority Data
Aug. 24, 2022 (JP) 2022-133468

(51) Int. Cl. C12M 1/00

(52) U.S. Cl. CPC C12M 47/02

(57) ABSTRACT

A density adjusting apparatus according to an embodiment of the present disclosure includes a flow path structure and processing circuitry. The flow path structure includes a main flow path and at least one branch flow path. A particle suspension flows into the main flow path. The one or more branch flow paths branch from the main flow path and are provided with one or more concentration valves related to concentrating the particle suspension. The processing circuitry obtains density information indicating a density of particles contained in the particle suspension and goal information indicating a goal density of the particles contained in output liquid caused to flow out of the main flow path. The processing circuitry controls opening and closing of the concentration valves, on the basis of the density information and the goal information.

### CROSS-REFERENCE TO RELATED APPLICATIONS

This application is based upon and claims the benefit of priority from Japanese Patent Application No. 2022-133468, filed on Aug. 24, 2022; the entire contents of which are incorporated herein by reference.

### FIELD

Embodiments described herein relate generally to a density adjusting apparatus and a density-adjusted liquid producing method.

### BACKGROUND

When particles such as cells are handled so as to perform cell processing and/or a biological test using the cells, it has conventionally been required to adjust the density of the cells in a cell suspension. Generally speaking, the majority of methods for adjusting a cell density are methods using centrifugal force. When centrifugal force is used, it is necessary to provide a centrifugal unit, and it is necessary to remove supernatant derived from centrifugation. For this reason, an apparatus (hereinafter, “density adjusting apparatus”) configured to adjust such a cell density can be expensive and have a complicated structure. Consequently, density adjusting apparatuses using centrifugal force are not susceptible to miniaturization.

Further, among the methods for adjusting a cell density, methods using filtering as a principle thereof involve loss of particles, i.e., cells. When the cells on a membrane serving as a filtering member are to be collected for the purpose of reducing such loss, it is necessary to perform, for example, a collecting operation such as suction and discharging. In that situation, a density adjusting apparatus using such a filtering process becomes complicated and is not susceptible for miniaturization. Further, although techniques have been developed so as to concentrate the density of particles in a micro flow path, because those methods generally involve passive concentration, a problem remains where the concentration ratio of the cells is dependent on the shape of a flow path, which makes it difficult to adjust the cell density to be a desired density.

### BRIEF DESCRIPTION OF THE DRAWINGS

FIG. 1 is a diagram illustrating an example of a density adjusting apparatus according to an embodiment of the present disclosure;

FIG. 2 is a drawing illustrating an example of a flow path structure according to the embodiment;

FIG. 3 is a drawing according to the embodiment illustrating an example in which a thermoresponsive polymer is used in one of a plurality of concentration valves;

FIG. 4 is a drawing according to the embodiment illustrating an example of a relationship between a part of the flow path structure and the thermoresponsive polymer;

FIG. 5 is a drawing according to the embodiment illustrating an example of opening and closing of a concentration valve, while an actuator is used as an example of the concentration valves;

FIG. 6 is a drawing according to the embodiment illustrating an example in which the concentration valve is structured with a screw and a driving mechanism configured to drive rotation of the screw;

FIG. 7 is a drawing according to the embodiment illustrating an example in which the concentration valve is structured with an elastic material and a driving mechanism configured to drive the elastic material;

FIG. 8 is a drawing according to the embodiment illustrating an example of a flow path connection in relation to the flow path structure, a particle suspension input unit, a transit liquid collecting unit, and an output liquid collecting unit;

FIG. 9 presents an example of a flowchart indicating an example of a procedure in a density adjusting process according to the embodiment;

FIG. 10 is a drawing illustrating an example of a flow path structure according to a modification example of the embodiment;

FIG. 11 is a drawing according to a first application example of the embodiment illustrating an example of a flow path structure capable of carrying out dilution; and

FIG. 12 is a drawing according to a second application example of the embodiment illustrating an example of a flow path structure capable of realizing a desired density by mixing together suspensions respectively containing two types of particles.

### DETAILED DESCRIPTION

A density adjusting apparatus according to an embodiment includes a flow path structure and processing circuitry. The flow path structure includes a main flow path and at least one branch flow path. Liquid (or a particle suspension) flows into the main flow path. The one or more branch flow paths branch from the main flow path and are provided with one or more concentration valves related to concentrating the particle suspension. The processing circuitry is configured to obtain density information indicating a density of particles contained in the particle suspension and goal information indicating a goal density of the particles contained in output liquid caused to flow out of the main flow path. The processing circuitry is configured to control opening and closing of the concentration valves, on the basis of the density information and the goal information.

Examples of a density adjusting apparatus and a density-adjusted liquid producing method will be explained below, with reference to the accompanying drawings. In the following embodiments, some of the elements referred to by using the same reference characters are assumed to perform the same operations, and duplicate explanations thereof will be omitted as appropriate. Further, the description of each of the embodiments is, in principle, similarly applicable to modification examples, application examples, and the like.

### Embodiments

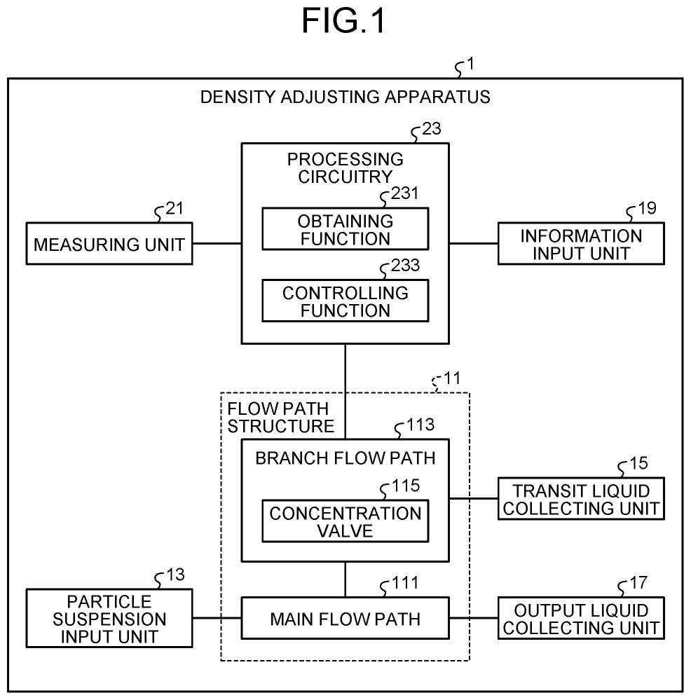

FIG. 1 is a diagram illustrating an example of a density adjusting apparatus 1 according to an embodiment of the present disclosure. As illustrated in FIG. 1, the density adjusting apparatus 1 includes a flow path structure 11, a particle suspension input unit 13, a transit liquid collecting unit 15, an output liquid collecting unit 17, an information input unit 19, a measuring unit 21, and processing circuitry 23. In another example, the information input unit 19 and the measuring unit 21 may be provided outside the density adjusting apparatus 1. In that situation, the density adjusting apparatus 1, the information input unit 19, and the measuring unit 21 structure a particle density adjusting system.

The flow path structure 11 includes a main flow path 111 and at least one branch flow path 113. The main flow path 111 and the branch flow path 113 are each a micro flow path having a diameter of micrometer order. The particles contained in liquid (a particle suspension) are assumed to be cells, for example. Further, the diameter of the branch flow path 113 is smaller than the diameter of the main flow path 111. For example, when the particles in liquid to be concentrated are cells, it is assumed that the diameter of the branch flow path 113 is smaller than the diameter of the cells. In other words, the branch flow path 113 has a diameter that prevents the cells (the particles contained in the liquid) from passing therethrough. As a result, transiting through the branch flow path 113 is the liquid. The diameters of the main flow path 111 and the branch flow path 113 are set in advance so that the ratio between a flow volume in the main flow path 111 and a flow volume in the branch flow path 113 is 8:1, for example. However, the ratio does not necessarily have to be 8:1 and may arbitrarily be set.

In the following sections, to explain a specific example, it is assumed that two or more branch flow paths 113 branch from the main flow path 111. Flowing into the main flow path 111 is liquid (hereinafter, “input liquid”) input to the density adjusting apparatus 1 via the particle suspension input unit 13. Liquid (hereinafter, “output liquid”) which has transited through the main flow path 111 and of which the density has been adjusted flows out to the collecting unit 17. The plurality of branch flow paths 113 are provided with a plurality of concentration valves 115 related to concentrating the suspension, respectively. Liquid (hereinafter, “transit liquid”) that has transited through the branch flow paths 113 flows out to the transit liquid collecting unit 15.

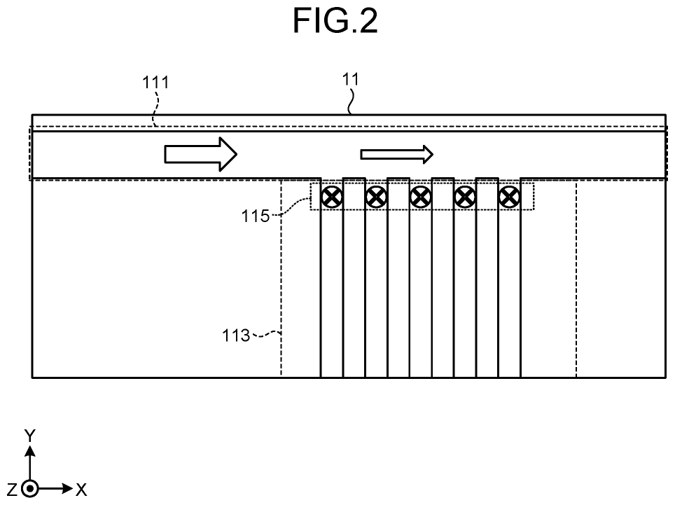

FIG. 2 is a drawing illustrating an example of the flow path structure 11. In FIG. 2, the vertical direction corresponds to a Z direction, whereas an X direction and a Y direction correspond to two directions orthogonal to each other on a horizontal plane. As illustrated in FIG. 2, in the main flow path 111, it is assumed that the input liquid flows along the X direction. Although FIG. 2 indicates that the quantity of the branch flow paths 113 is five, the quantity is not limited to this example and may be ten to twenty, for instance. In those situations, it is possible to concentrate the density of the particles in the output liquid, so that the density becomes 1 to 6.7 times higher than the density of the particles in the input liquid, for example.

The plurality of concentration valves 115 are provided for the plurality of branch flow paths 113, respectively. Each of the plurality of concentration valves 115 opens and closes under control of a controlling function 233 of the processing circuitry 23. The opening and closing of the plurality of concentration valves 115 correspond to, for example, an opening or closing degree of each of the plurality of concentration valves 115, each of the plurality of concentration valves 115 fully closing, or each of the plurality of concentration valves 115 fully opening/closing. The plurality of concentration valves 115 are structured with a material and a mechanism that enable the opening and closing thereof according to control signals output from the controlling function 233. In the following sections, examples of installing each of the plurality of concentration valves 115 will be explained.

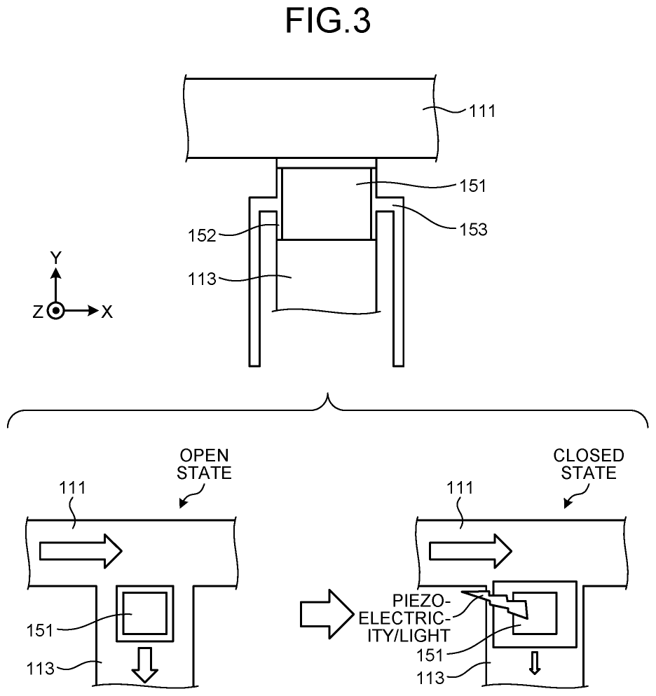

FIG. 3 is a drawing illustrating an example in which a thermoresponsive polymer 151 is used in one of the plurality of concentration valves 115. In other words, as a material structuring the plurality of concentration valves 115, the thermoresponsive polymer 151 is used. The thermoresponsive polymer 151 may be realized by using a thermoresponsive gel, for example. To the thermoresponsive polymer 151, a thin film electrode 152 is attached as a mechanism that enables the opening and closing according to the control signals output from the controlling function 233. In the following sections, an example will be explained in which poly(N-isopropylacrylamide) (hereinafter, “PNIPAM”) is used as an example of the thermoresponsive polymer.

As illustrated in FIG. 3, the PNIPAM 151 is provided with a metal conductor 153 that transfers heat related to controlling movements of the PNIPAM 151, as a mechanism structuring the one of the plurality of concentration valves 115. The conductor 153 is electrically connected to the thin film electrode 152 structured with gold and/or chrome or the like, for example. Further, one conductor 153 is independently provided for each of the plurality of concentration valves 115 so as to individually control each of the plurality of concentration valves 115, i.e., so as to be able to independently control each of the plurality of concentration valves 115 provided in correspondence with the plurality of branch flow paths 113.

As illustrated in FIG. 3, when no heat is supplied to the PNIPAM 151, the concentration valve 115 is in an open state, for example. In that situation, the liquid flows out to the branch flow path 113 from the main flow path 111. On the contrary, as illustrated in FIG. 3, when heat is supplied to the PNIPAM 151, the thin film electrode 152, which is a metal base, is heated. As a result, the PNIPAM (thermoresponsive resin) 151 expands and becomes hardened so as to close the branch flow path 113. Thus, a close state of the branch flow path 113 is realized.

In the description above, the thermoresponsive polymer 151 is used as the concentration valve 115; however, means for realizing the concentration valves 115 are not limited to this example. For instance, it is also acceptable to use a piezoelectric element that expands with application of voltage or a light-responsive element that expands upon receipt of light, as each of the concentration valves 115. In those situations, as the conductor 153, an electrically conductive member related to the application of the voltage or an optical cable that transmits the light may be used.

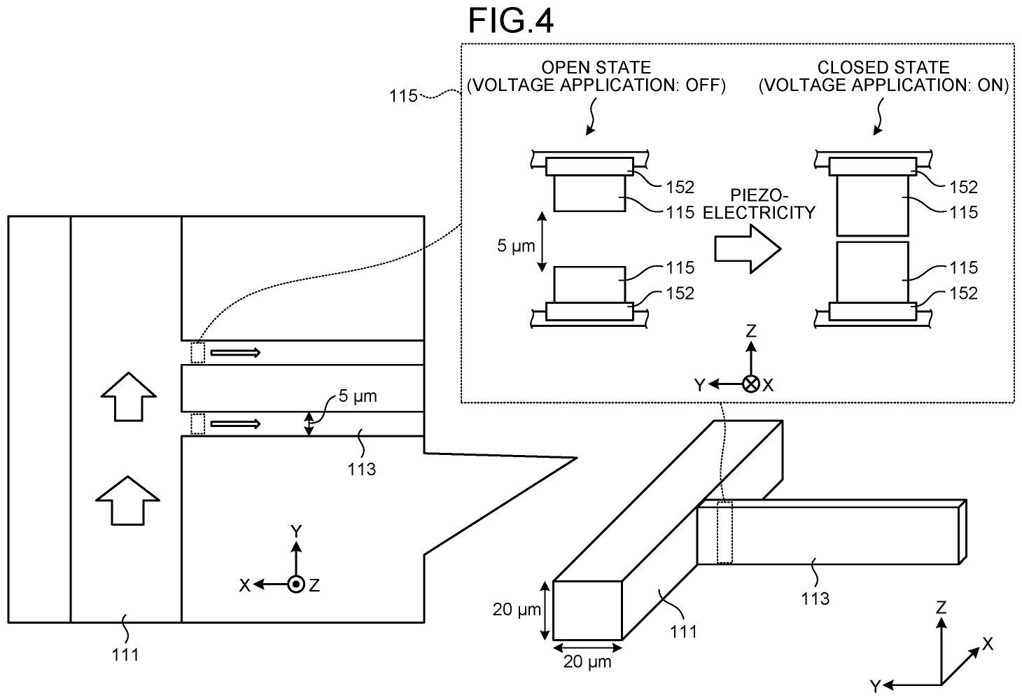

FIG. 4 is a drawing illustrating an example of a relationship between a part of the flow path structure 11 and the thermoresponsive polymer 151. FIG. 4 indicates an exemplary configuration in which 10-μm particles are concentrated, while a cross-sectional shape of the main flow path 111 is a quadrangle of 20 μm by 20 μm, whereas the diameter of each of the branch flow paths 113 is 5 μm. The dimensions of the diameters and the cross-sectional shape are merely examples, and possible embodiments are not limited to these examples. For instance, the diameters of the main flow path 111 and the branch flow paths 113 may be set on the basis of the size of the particles to be concentrated, at the time of designing the flow path structure 11. Further, the quantity of the branch flow paths 113 may be determined according to a density range for desired concentration.

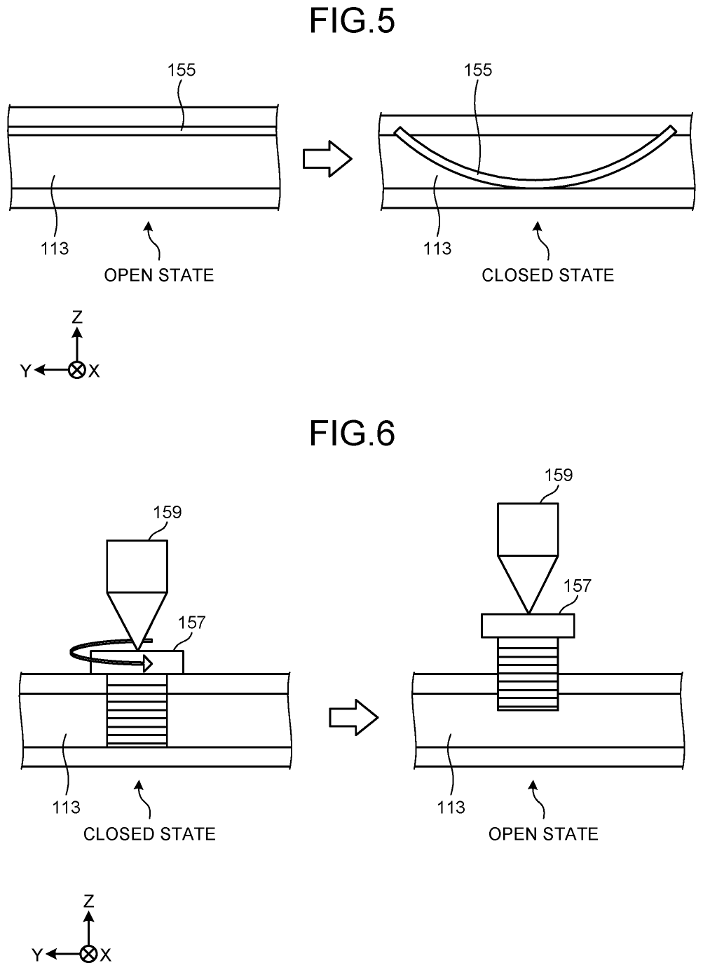

FIG. 5 is a drawing illustrating an example of the opening and closing of the concentration valve 115, while an actuator 155 is used as an example of the concentration valves 115. As illustrated in FIG. 5, while no voltage is applied to the actuator 155, the concentration valve 115 is in an open state, for example. On the contrary, when voltage is applied to the actuator 155 as illustrated in FIG. 5, the actuator 155 changes the shape thereof and closes the branch flow path 113. Thus, a closed state of the branch flow path 113 is realized.

FIG. 6 is a drawing illustrating an example in which the concentration valve 115 is structured with a screw 157 and a driving mechanism 159 configured to drive rotation of the screw 157. For example, the driving mechanism 159 may be structured with a motor. The driving mechanism 159 is configured to rotate the screw 157 under control of the controlling function 233. As a result, an open state, an opening degree, a closing degree, and a closed state of the branch flow path 113 are realized.

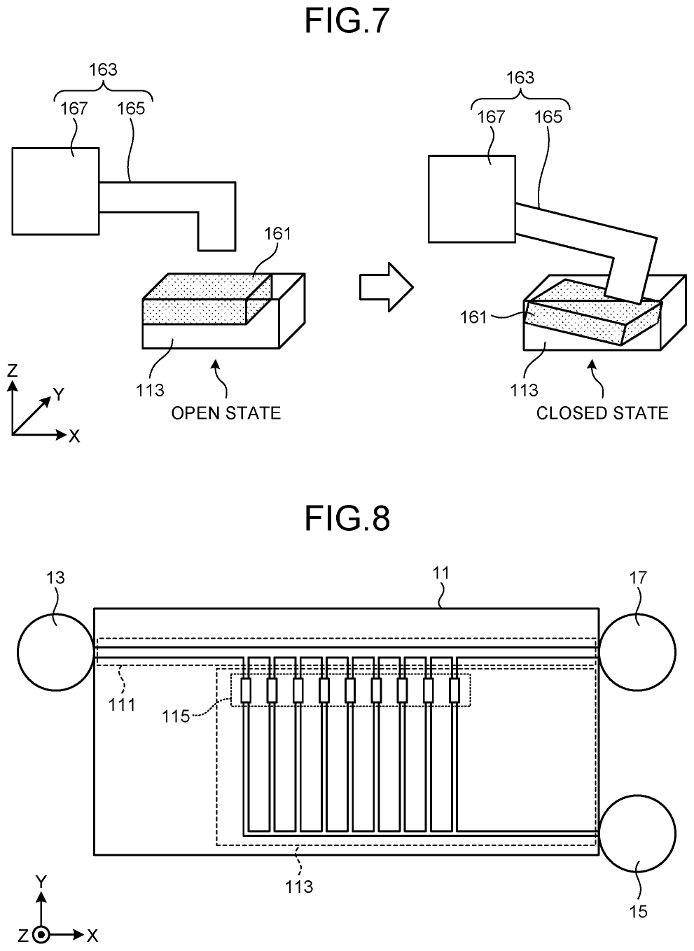

FIG. 7 is a drawing illustrating an example in which the concentration valve 115 is structured with an elastic material 161 and a driving mechanism 163 configured to drive the elastic material 161. The driving mechanism 163 may be structured with a frame 165 configured to press the elastic material 161 and a motor 167 configured to drive the frame 165, for example. The elastic material 161 may be realized by using a known elastic body or a blister material capable of closing the branch flow path 113. The driving mechanism 163 structured with the frame 165 and the motor 167 corresponds to a mechanism configured to mechanically distort the elastic material 161.

As illustrated in FIG. 7, due to rotation of the motor 167 under control of the controlling function 233, the frame 165 presses the elastic material 161. In other words, when a signal instructing to distort the elastic material 161 is output to the motor 167 as a control signal, the frame 165 presses the elastic material 161 as being driven by the motor 167. As a result, a closed state of the branch flow path 113 is realized. Further, when the frame 165 leaves the elastic material 161 as a result of the motor 167 rotating backwards under control of the controlling function 233, the elastic material 161 returns to the original shape thereof with elastic force thereof. As a result, an open state of the branch flow path 113 is realized. Possible means for realizing the concentration valves 115 are not limited to those described above. It is also acceptable to realize the concentration valves 115 by using other known methods such as torque-based valves.

FIG. 8 is a drawing illustrating an example of a flow path connection related to the flow path structure 11, the particle suspension input unit 13, the transit liquid collecting unit 15, and the output liquid collecting unit 17. As illustrated in FIG. 8, the particle suspension input unit 13 is provided on the upstream side of the main flow path 111. The transit liquid collecting unit 15 is provided on the downstream side of the branch flow path 113. The output liquid collecting unit 17 is provided on the downstream side of the main flow path 111.

The particle suspension input unit 13 is configured to cause the liquid to flow into the main flow path 111. The particle suspension input unit 13 is realized by using, for example, a connector that connects a tube connected to a pump configured to pump out the input liquid, to the density adjusting apparatus 1. Alternatively, the particle suspension input unit 13 may be realized by using, for example, a pump configured to pump out the particle suspension stored in a tank to the density adjusting apparatus 1.

The transit liquid collecting unit 15 is, more specifically, provided on the downstream side of a merging point where the plurality of branch flow paths 113 merge together. The transit liquid collecting unit 15 is configured to collect the transit liquid that has transited through the branch flow paths 113. The transit liquid collecting unit 15 is realized, for example, by using a tank (a transit liquid collecting tank), a test tube, or the like capable of collecting the transit liquid.

The output liquid collecting unit 17 is configured to collect the output liquid having a goal density. The output liquid collecting unit 17 is realized by using a tank (an output liquid collecting tank), a test tube, or the like capable of collecting the output liquid.

The information input unit 19 is configured to receive an input of goal information indicating the goal density of the particles contained in the output liquid. More specifically, the information input unit 19 is configured to receive the density of the output liquid according to an instruction from the user, as the goal information. For example, the information input unit 19 is realized by using an input interface. The input interface is configured to receive various types of instructions and inputs of information from the user. The input interface is realized by using, for example, a trackball, a switch button, a mouse, a keyboard, a touchpad on which input operations can be performed by touching an operation surface thereof, a touch screen in which a display screen and a touchpad are integrally formed, contactless input circuitry using an optical sensor, audio input circuitry, and/or the like. The input interface is connected to the processing circuitry 23 and is configured to convert the input operations received from the user into electrical signals and to output the electrical signals to the processing circuitry 23.

However, the input interface of the present disclosure does not necessarily have to include physical operation component parts such as a mouse, a keyboard, and/or the like. For instance, possible examples of the input interface include electrical signal processing circuitry configured to receive an electrical signal corresponding to an input operation from an external input mechanism provided separately from the density adjusting apparatus 1 and to output the electrical signal to the processing circuitry 23.

The measuring unit 21 is configured to measure the density of the particles contained in the input liquid on the upstream side of the most upstream branch point among the branch points between the main flow path 111 and the branch flow paths 113. In this situation, the measuring unit 21 may measure the density of the particles contained in the input liquid on the upstream side of the particle suspension input unit 13. The measuring unit 21 is realized by using a density sensor, for example.

In addition, the measuring unit 21 may further measure a physical property and/or a chemical property of the input liquid. Examples of the physical property include viscosity of the input liquid. Examples of the chemical property include a hydrogen ion index (pH). In that situation, the measuring unit 21 is structured with a density sensor and a viscosity sensor configured to measure the viscosity (and/or a pH sensor). Because it is possible to use known sensors as the density sensor, the viscosity sensor, and the pH sensor, explanations thereof will be omitted. Further, the measuring unit 21 may be continuous with the main flow path 111 in the flow path structure 11 or may be provided separately from the main flow path 111.

The processing circuitry 23 is realized by using, as hardware resources thereof, a processor and one or more memory elements such as a Read-Only Memory (ROM), a Random Access Memory (Memory), and/or the like. The term “processor” denotes, for example, a Central Processing Unit (CPU), a Micro Processing Unit (MPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). The processing circuitry 23 corresponds to a processing unit.

The processing circuitry 23 includes an obtaining function 231 and the controlling function 233. The processing circuitry 23 realizing the obtaining function 231 and the controlling function 233 corresponds to an obtaining unit and a controlling unit, respectively. The functions such as the obtaining function 231 and the controlling function 233 are stored in a memory of the processing circuitry 23 in the form of computer-executable programs. For example, the processing circuitry 23 is configured to realize the functions corresponding to the programs, by reading and executing the programs from a memory 125. In other words, the processing circuitry 23 that has read the programs has the obtaining function 231 and the controlling function 233, or the like.

Although the example was explained above in which the “processor” reads and executes the programs corresponding to the functions from the memory, possible embodiments are not limited to this example. When the processor is a CPU, for example, the processor realizes the functions by reading and executing the programs saved in the memory. In contrast, when the processor is an ASIC, for example, instead of having the programs saved in the memory, the functions are directly incorporated in the circuitry of the processor as logic circuitry. Further, the processors in the present embodiments do not each necessarily have to be structured as a single piece of circuitry. It is also acceptable to structure one processor by combining together a plurality of pieces of independent circuitry, so as to realize the functions thereof. Furthermore, although the example was explained in which the single piece of storage circuitry has stored therein the programs corresponding to the processing functions, it is also acceptable to provide a plurality of pieces of storage circuitry in a distributed manner, so that the processing circuitry 23 reads corresponding programs from the individual pieces of storage circuitry.

By employing the obtaining function 231, the processing circuitry 23 is configured to obtain density information indicating a density of the particles contained in the input liquid and the goal information indicating the goal density of the particles contained in the output liquid caused to flow out of the main flow path 111. More specifically, the obtaining function 231 is configured to obtain the goal information from the information input unit 19. The obtaining function 231 is configured to obtain the density information from the measuring unit 21.

By employing the controlling function 233, the processing circuitry 23 is configured to control the opening and closing of the concentration valves 115, on the basis of the density information and the goal information. More specifically, when the density information is known to be a prescribed value, the controlling function 233 is configured to determine a quantity of the concentration valves (hereinafter, “valve quantity”) to be opened among the plurality of concentration valves 115, so that the density of the particles in the output liquid approximates the goal density, in accordance with the density (the goal information) desired by the user. For example, the controlling function 233 may be configured to determine the valve quantity by matching the density information and the goal information against a correspondence table (hereinafter, “density open quantity correspondence table”) indicating valve quantities kept in correspondence with density values of the input liquid caused to flow in by the particle suspension input unit 13 and goal density values of the output liquid. In the density open quantity correspondence table, the valve quantities are set in such a manner that, while the density of the particles in the input liquid is the same, the higher a targeted density is, the larger is the valve quantity. Preferably, the density open quantity correspondence table is set in such a manner that the density of the particles in the output liquid becomes substantially equal to the goal density.

Further, in addition to determining the valve quantity, the controlling function 233 may be configured to determine an extent (a valve opening degree) by which each of the plurality of concentration valves 115 is opened. As another example, in addition to determining the valve quantity, the controlling function 233 may be configured to determine an extent (a valve closing degree) by which each of the plurality of concentration valves 115 is closed.

In yet another example, the controlling function 233 may be configured to determine a valve quantity by using a formula (hereinafter, “open quantity calculation formula”) for calculating the valve quantity with inputs of the density information and the goal information, in place of the density open quantity correspondence table. In yet another example, the controlling function 233 may be configured to determine a quantity of the valves to be opened, in accordance with the difference in density between the density information and the goal information.

In yet another example, the controlling function 233 may be configured to calculate a concentration ratio of the density per branch flow path. In this situation, the concentration ratio may empirically be determined in advance, so as to be stored into a memory of the processing circuitry 23. In that situation, the controlling function 233 may be configured to determine the quantity of the concentration valves to be opened, on the basis of the density information, the goal information, and the concentration ratio per branch flow path. More specifically, for example, the controlling function 233 may be configured to determine the quantity of the concentration valves to be opened, by taking the concentration ratio per branch flow path into account with the open quantity calculation formula.

In another example, the controlling function 233 may be configured to determine the quantity of the concentration valves to be opened on the basis of a cross-sectional area ratio between the main flow path 111 and the branch flow paths 113 stored in a memory in advance and the difference in density between the density information and the goal information. In that situation, the controlling function 233 is configured to determine the quantity of the concentration valves to be opened, by using either a correspondence table or a calculation formula obtained by adding the cross-sectional area ratio to either the density open quantity correspondence table or the open quantity calculation formula.

In yet another example, the controlling function 233 may be configured to determine the quantity of the concentration valves to be opened, by further using a physical property of the input liquid (e.g., viscosity of the input liquid) measured by the measuring unit 21. In that situation, the controlling function 233 is configured to determine the quantity of the concentration valves to be opened, by using either a correspondence table or a calculation formula obtained by adding the physical property (and/or a chemical property) to either the density open quantity correspondence table or the open quantity calculation formula.

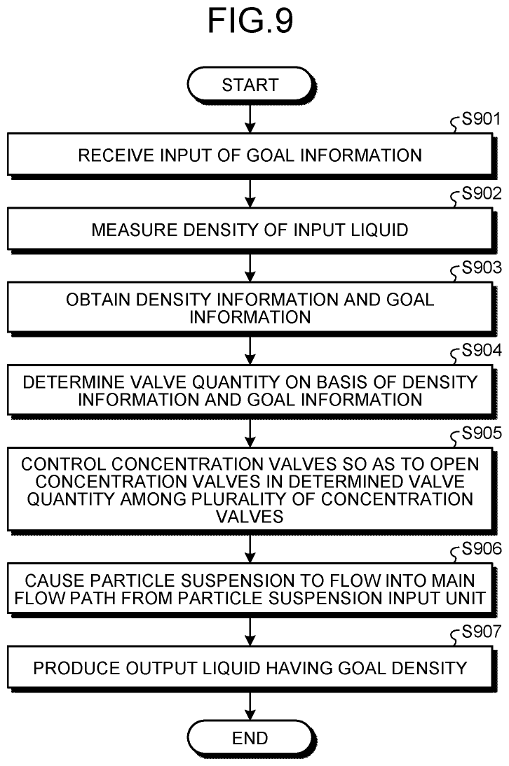

The constituent elements of the density adjusting apparatus 1 have thus been explained. Next, a procedure in a process (hereinafter, “density adjusting process”) in which the density adjusting apparatus 1 produces the output liquid having the goal particle density from the input liquid will be explained. A method for realizing the density adjusting process may be referred to as a density-adjusted liquid producing method. FIG. 9 illustrates an example of a flowchart indicating an example of the procedure in the density adjusting process.

### Density Adjusting Process

### Step S901:

According to a user instruction, the information input unit 19 receives an input of a goal density for the output liquid.

### Step S902:

The measuring unit 21 measures the density of the input liquid caused to flow into the main flow path 111 from the particle suspension input unit 13. In this situation, the measuring unit 21 may measure a physical property (and/or a chemical property) of the input liquid caused to flow into the main flow path 111 from the particle suspension input unit 13.

### Step S903:

By employing the obtaining function 231, the processing circuitry 23 obtains the density information from the measuring unit 21. In addition, the obtaining function 231 obtains the goal information from the information input unit 19.

### Step S904:

By employing the controlling function 233, the processing circuitry 23 determines a valve quantity on the basis of the density information and the goal information. For example, the controlling function 233 determines the valve quantity, by applying the density information and the goal information to either the density open quantity correspondence table or the open quantity calculation formula. In this situation, when a physical property such as the viscosity of the input liquid has been measured, the controlling function 233 may determine the valve quantity reflecting the measured physical property also. Further, depending on the structure of the plurality of concentration valves 115, the controlling function 233 may determine opening degrees of the valves or closing degrees of the valves, in addition to the valve quantity.

### Step S905:

By employing the controlling function 233, the processing circuitry 23 controls the plurality of concentration valves 115, so as to open the valves in the determined quantity. For example, when the concentration valves 115 are each structured with the thermoresponsive polymer 151 and the thin film electrode 152, the controlling function 233 applies voltage to the thin film electrode 152 as a control signal. In another example, when the concentration valves 115 are each structured with the elastic material 161 and the driving mechanism 163, the controlling function 233 outputs a signal for distorting the elastic material 161 to the motor 167 of the driving mechanism 163, as a control signal. In an example, when the valve opening degrees or the valve closing degrees were determined at step S904, the controlling function 233 controls the plurality of concentration valves 115 so as to realize the valve opening degrees or the valve closing degrees.

### Step S906:

The particle suspension input unit 13 causes the particle suspension to flow into the main flow path 111. In this situation, the particles (e.g., the cells) contained in the input liquid having flowed into the main flow path 111 reach the output liquid collecting unit 17, together with the output liquid. Further, a part of the liquid of the input liquid flows into the branch flow paths 113 via the open concentration valves and reaches the transit liquid collecting unit 15.

### Step S907:

The output liquid having the goal density reaches the output liquid collecting unit 17. As a result of the processes described above, the output liquid having the goal density has been produced.

The density adjusting apparatus 1 according to the embodiment described above has the flow path structure 11 including the main flow path 111 into which the input liquid flows and at least one branch flow path 113 that branches from the main flow path 111 and is provided with at least one concentration valve 115 related to concentrating the input liquid. The density adjusting apparatus 1 is configured to obtain the density information indicating the density of the particles contained in the input liquid flowing into the main flow path 111 and the goal information indicating the goal density of the particles contained in the output liquid caused to flow out of the main flow path 111 and is configured to control the opening and closing of the concentration valve 115 on the basis of the density information and the goal information. For example, when controlling the opening and closing of the concentration valve 115, the density adjusting apparatus 1 according to the present embodiment is configured to control one of: an opening or closing degree of the concentration valve 115; and fully opening/closing the concentration valve 115.

With these arrangements, the density adjusting apparatus 1 according to the embodiment is able to conveniently adjust the density of the input liquid without the need to have a complicated configuration in the flow path structure 11 or the like and without causing a particle loss in the micro flow paths. For example, when the ratio between a flow volume in the main flow path 111 and a flow volume in the branch flow path 113 is 8:1, while the quantity of the branch flow paths 113 is ten to twenty, the density adjusting apparatus 1 according to the embodiment is able to concentrate the density of the particles in the output liquid, so that the density becomes 1 to 6.7 times higher than the density of the particles in the input liquid.

Further, the density adjusting apparatus 1 according to the embodiment is configured to collect the transit liquid that has transited through the branch flow paths 113. As a result, by using the density adjusting apparatus 1 according to the embodiment, it is possible to discard the transit liquid as waste liquid or to use the transit liquid in an analysis or the like. For example, when the transit liquid is culture medium supernatant of the cells, it is possible to use the collected transit liquid for analyzing nucleic acid, proteins, or extracellular vesicles. In that situation, by using the density adjusting apparatus 1 according to the present embodiment, it is possible to utilize the collected transit liquid for the analysis, by taking the transit liquid out of the input liquid in an amount that is necessary.

Further, the density adjusting apparatus 1 according to the embodiment is configured to measure the density of the particles in the input liquid on the upstream side of the most upstream branch point among the branch points between the main flow path 111 and the branch flow paths 113 and is configured to receive the input of the density of the output liquid desired by the user. Accordingly, the density adjusting apparatus 1 according to the present embodiment is able to produce the output liquid having the density desired by the user. Further, the density adjusting apparatus 1 according to the embodiment may be configured to measure a physical property and/or a chemical property of the input liquid. In that situation, the density adjusting apparatus 1 according to the embodiment is able to produce the output liquid having an improved precision level for the density, by controlling the opening and closing of the concentration valves 115 while further using the physical property and/or the chemical property of the input liquid.

Further, the concentration valves 115 of the density adjusting apparatus 1 according to the embodiment are structured with the material and the mechanism that enable the opening and closing thereof, according to the control signal output from the controlling function 233. For example, the material of the concentration valves 115 according to the present embodiment may be the thermoresponsive polymer 151, for example. In that situation, to the thermoresponsive polymer 151, the thin film electrode 152 may be attached as the mechanism, so that the density adjusting apparatus 1 according to the present embodiment applies voltage to the thin film electrode 152 as the control signal. With these arrangements, when the density adjusting apparatus 1 according to the embodiment is used, it is possible to open and close the concentration valves 115 through a piezoelectric operation or the like. It is therefore possible to realize structuring and controlling the concentration valves 115 in a relatively simple manner.

Further, with respect to the concentration valves 115 each structured with the elastic material 161 and the mechanism (driving mechanism) 163 configured to mechanically distort the elastic material 161, the density adjusting apparatus 1 according to the embodiment is configured to output the signal for distorting the elastic material 161 to the mechanism 163, as the control signal. With this arrangement, by using the density adjusting apparatus 1 according to the embodiment, it is possible to control the opening and closing of the concentration valves 115 at a low cost and conveniently, by applying the physical pressure to the branch flow paths 113. Alternatively, the concentration valves 115 of the density adjusting apparatus 1 according to the embodiment may each be structured with the screw 157 and the driving mechanism 159 configured to drive rotation of the screw 157. In that situation, the density adjusting apparatus 1 according to the embodiment is able to finely adjust the density of the output liquid, by adjusting the opening/closing degrees of the branch flow paths 113. Consequently, by using the density adjusting apparatus 1 according to the embodiment, it is possible to realize the concentration process with a high level of precision.

As explained above, by using the density adjusting apparatus 1 according to the present embodiment, it is possible to produce a cell liquid having the desired density, by using, for example, the plurality of branch flow paths 113 provided with the concentration valves (micro flow path valves) 115 structured with the thermoresponsive polymer 151 or the like. Consequently, by using the density adjusting apparatus 1 according to the present embodiment, it is possible, within the micro flow paths, to conveniently adjust the particle density to be the desired density, while minimizing loss of the cells. In addition, by using the density adjusting apparatus 1 according to the present embodiment, it is possible to adjust the particle density to be the desired density in a compact scale.

### Modification Examples

A modification example of the present embodiment is configured so as to decrease the cross-sectional area of the main flow path 111 along the flowing direction of the input liquid, on the upstream side of the most upstream branch point among the branch points between the main flow path 111 and the branch flow paths 113. FIG. 10 is a drawing illustrating an example of a flow path structure 101 according to the present modification example. As illustrated in FIG. 10, the cross-sectional area of the main flow path 111 is varied so as to decrease along the flowing direction of the liquid, in the main flow path 111 in an upstream part of the most upstream branch point among the branch points between the main flow path 111 and the branch flow paths 113.

Further, as illustrated in FIG. 10, the angles of the branch flow paths 113 formed with the main flow path 111 with respect to the flowing direction of the input liquid are acute angles. With this arrangement, in the density adjusting apparatus 1 according to the present modification example, it is possible to increase the flow volume in the sections where the main flow path 111 branches into the branch flow paths 113. It is therefore possible to realize the process of concentrating the density of the input liquid more efficiently. Because the other advantageous effects of the present modification example are the same as those of the embodiment, explanations thereof will be omitted.

### First Application Example

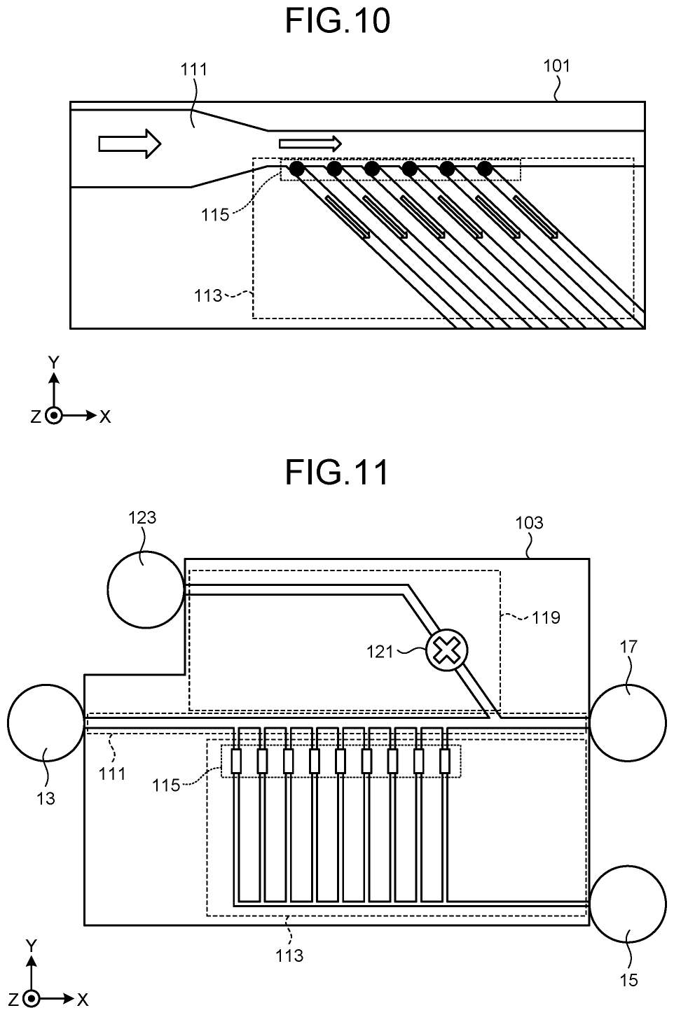

In a first application example of the embodiment, to adjust the density of the output liquid, the density of the input liquid is diluted, for example. In the following sections, to explain a specific example, it will be assumed that a flow path structure 103 in the present application example is configured as illustrated in FIG. 11, for instance. FIG. 11 is a drawing illustrating the example of the flow path structure 103 capable of carrying out the dilution.

As illustrated in FIG. 11, the flow path structure 103 has a merging flow path 119 that merges with the main flow path 111 on the downstream side of the most downstream branch point among the branch points between the main flow path 111 and the branch flow paths 113. The merging flow path 119 is provided with a dilution valve 121. In other words, the dilution valve 121 is a valve provided for the purpose of diluting the particle suspension on the downstream side of the most downstream branch point. Because it is possible to apply a structure similar to that in the embodiment to the dilution valve 121, explanations thereof will be omitted. Opening and closing of the dilution valve 121 corresponds, for example, to an opening degree or a closing degree of the dilution valve 121.

As illustrated in FIG. 11, the output liquid collecting unit 17 is provided on the downstream side of the merging point between the merging flow path 119 and the main flow path 111. Further, a diluted particle suspension input unit 123 is provided on the upstream side of the merging flow path 119. The diluted particle suspension input unit 123 is configured to cause diluted liquid having lower density than the input liquid to flow into the merging flow path 119. For example, when the input liquid contains cells, the diluted liquid corresponds to a liquid (hereinafter, “culture medium liquid”) serving as a culture medium of the cells. However, the diluted liquid does not necessarily have to be a culture medium liquid and may be saline, for example.

By employing the controlling function 233, the processing circuitry 23 is configured to control the opening and closing of the dilution valve 121, on the basis of the density information and the goal information. More specifically, when the density information is known to be a prescribed value, the controlling function 233 is configured to determine an extent (hereinafter, “open amount”) by which the dilution valve 121 is to be opened, in accordance with the density (the goal information) desired by the user. For example, the controlling function 233 may be configured to determine the open amount by matching the density information and the goal information against a correspondence table (hereinafter, “dilution correspondence table”) indicating open amounts kept in correspondence with density values of the input liquid and density values of the liquid input by the user.

In another example, in place of the dilution correspondence table, the controlling function 233 may be configured to determine the open amount by using a formula (hereinafter, “dilution calculation formula”) for calculating the open amount with inputs of the density information and the goal information. In yet another example, the controlling function 233 may be configured to determine the open amount, in accordance with the difference in density between the density information and the goal information.

In the density adjusting apparatus 1 according to the first application example of the embodiment, the flow path structure 103 further includes, on the downstream side of the most downstream branch point among the branch points between the main flow path 111 and the branch flow paths 113, the merging flow path 119 that merges with the main flow path 111 and is provided with the dilution valve 121 for diluting the particle suspension on the downstream side. The output liquid collecting unit 17 is provided on the downstream side of the merging point between the merging flow path 119 and the main flow path 111. The controlling function 233 is configured to control the opening and closing of the dilution valve 121 on the basis of the density information and the goal information.

With these arrangements, the density adjusting apparatus 1 according to the first application example of the embodiment is able to concentrate and dilute the cells within the flow path structure 103. Further, when the screw-based structure illustrated in FIG. 6 is used, for example, as the dilution valve 121 of the density adjusting apparatus 1 according to the first application example, it is possible to realize arbitrary dilution as desired by the user. Because the other advantageous effects of the first application example are the same as those of the embodiment, explanations thereof will be omitted.

### Second Application Example

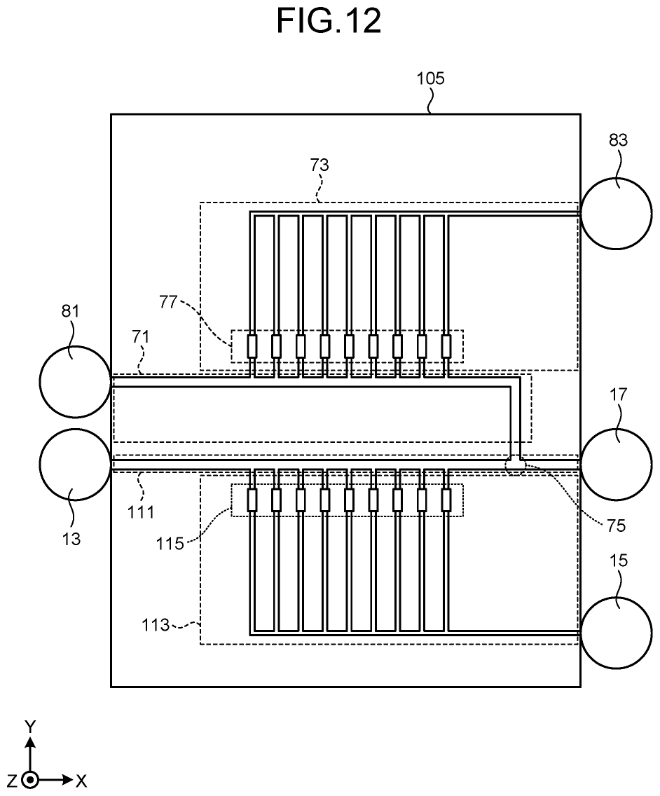

In a second application example, suspensions respectively containing a plurality of types of particles are mixed together to realize a density desired by the user. In the following sections, an example will be explained in which suspensions respectively containing two types of particles are mixed together. FIG. 12 is a drawing illustrating an example of a flow path structure 105 capable of realizing a desired density by mixing together the suspensions respectively containing two types of particles. As illustrated in FIG. 12, the flow path structure 105 in the present application example is obtained by connecting two structures together each being the flow path structure 11 illustrated in FIG. 8. Further, when suspensions respectively containing “n” types of particles (where n is a natural number of 3 or larger) are mixed together, the flow path structure 105 in the present application example is obtained by connecting “n” structures together each being the flow path structure 11 illustrated in FIG. 8.

As illustrated in FIG. 12, in addition to the elements of the flow path structure 11 illustrated in FIG. 8, the flow path structure 105 according to the present application example further includes an extra main flow path 71, at least one extra branch flow path 73, and a main flow merging part 75. Flowing into the extra main flow path 71 is a suspension containing particles different from those in the main flow path 111 illustrated in FIG. 8. The one or more extra branch flow paths 73 branch from the extra main flow path 71 and are provided with one or more extra concentration valves 77. Because the structures of the extra main flow path 71, the extra branch flow paths 73, and the extra concentration valves 77 are the same as those of the main flow path 111, the branch flow paths 113, and the concentration valves 115, respectively, explanations thereof will be omitted. The main flow merging part 75 corresponds to a merging part where the main flow path 111 merges with the extra main flow path 71, on the downstream side of the most downstream branch point among the branch points between the main flow path 111 and the branch flow paths 113.

As illustrated in FIG. 12, an extra particle suspension input unit 81 is provided on the upstream side of the extra main flow path 71. The extra particle suspension input unit 81 is configured to cause another suspension (hereinafter, “the other suspension”) to flow into the extra main flow path 71. An extra transit liquid collecting unit 83 is provided on the downstream side of the extra branch flow paths 73. More specifically, the extra transit liquid collecting unit 83 is provided on the downstream side of a merging point where the plurality of extra branch flow paths 73 merge together. The extra transit liquid collecting unit 83 is configured to collect transit liquid that has transited through the extra branch flow paths 73. The output liquid collecting unit 17 is provided on the downstream side of the main flow merging part 75. Because the structures of the extra particle suspension input unit 81 and the extra transit liquid collecting unit 83 are the same as those of the particle suspension input unit 13 and the transit liquid collecting unit 15, explanations thereof will be omitted.

The information input unit 19 is configured to receive an input of extra goal information indicating a goal density of other particles contained in the other output liquid caused to flow out of the extra main flow path 71.

On the upstream side of the most upstream branch point among the branch points between the extra main flow path 71 and the extra branch flow paths 73, the measuring unit 21 is configured to measure the density of the other particles contained in the other suspension caused to flow into the extra main flow path 71.

By employing the obtaining function 231, the processing circuitry 23 is configured to obtain extra density information and extra goal information. By employing the controlling function 233, the processing circuitry 23 is configured to control the opening and closing of the extra concentration valves 77, on the basis of the extra density information and the extra goal information. Because the process related to controlling the opening and closing of the extra concentration valves 77 can be comprehended by replacing the description about the particles contained in the input liquid in the explanations of the embodiment, with a description about the other particles contained in the other input liquid or the like, explanations thereof will be omitted.

In the density adjusting apparatus 1 according to the second application example of the embodiment, the flow path structure 105 further includes: the extra main flow path 71 different from the main flow path 111; at least one extra branch flow path 73 that branches from the extra main flow path 71 and is provided with at least one extra concentration valve 77; and the main flow merging part 75 where the main flow path 111 merges with the extra main flow path 71, on the downstream side of the most downstream branch point among the branch points between the main flow path 111 and the branch flow paths 113. The obtaining function 231 is configured to obtain the extra density information and the extra goal information. The controlling function 233 is configured to control the opening and closing of the extra concentration valve 77, on the basis of the extra density information and the extra goal information.

With these arrangements, by using the density adjusting apparatus 1 according to the second application example of the embodiment, when the user wishes to mix together the plurality of types of particle suspensions to realize the adjusted density, it is possible to mix together, for example, the two types of particles within the main flow path 111, so that the adjusted density is realized. The density adjusting apparatus 1 according to the second application example is effective in the situation where, for example, the user wishes to mix two types of cells while realizing an adjusted density. Because the other advantageous effects of the second application example are the same as those of the embodiment, explanations thereof will be omitted.

### Third Application Example

In a third application example, the concentration valves 115 are controlled on the basis of output density information indicating a density of the output liquid in the output liquid collecting unit 17 and the goal information. The control exercised on the opening and closing of the concentration valves 115 in the present application example corresponds to feedback control on the opening and closing of the concentration valves 115 based on the output density information and the goal information.

The measuring unit 21 is configured to measure the density of the output liquid caused to flow out of the main flow path 111. Alternatively, the measuring unit 21 may be configured to measure the density of the output liquid collected by the output liquid collecting unit 17.

By employing the obtaining function 231, the processing circuitry 23 is configured to obtain the output density information indicating the density of the output liquid. By employing the controlling function 233, the processing circuitry 23 is configured to control the concentration valves 115, on the basis of the output density information and the goal information. Because the control exercised on the opening and closing of the concentration valves 115 in the present application example is the same as that in the embodiment, except that the data used for the control is the output density information, explanations thereof will be omitted.

The density adjusting apparatus 1 according to the third application example of the embodiment is configured to further obtain the output density information indicating the density of the particles contained in the output liquid and to further control the opening and closing of the concentration valves 115 on the basis of the output density information and the goal information. With this arrangement, the density adjusting apparatus 1 according to the third application example is able to exercise the feedback control on the opening and closing of the concentration valves 115, by using the density of the output liquid. Consequently, the density adjusting apparatus 1 according to the third application example is able to produce the output liquid having the goal density with a higher level of precision. Because the other advantageous effects are the same as those of the embodiment, explanations thereof will be omitted.

When technical features of the present embodiment are realized as a density-adjusted liquid producing method, the density-adjusted liquid producing method includes: causing the particle suspension to flow into the main flow path 111 in the flow path structure 11 including the main flow path 111 into which the particle suspension flows and at least one branch flow path 113 that branches from the main flow path 111 and is provided with at least one concentration valve 115 related to concentrating the particle suspension; obtaining the density information indicating the density of the particles contained in the particle suspension and the goal information indicating the goal density of the particles contained in the output liquid caused to flow out of the main flow path 111; and producing the output liquid having the goal density from the particle suspension, by controlling the opening and closing of the concentration valve 115 on the basis of the density information and the goal information. Because the processing procedure and advantageous effects of the density-adjusted liquid producing method are the same as those of the embodiment, explanations thereof will be omitted.

According to at least one aspect of the embodiments and the like described above, it is possible to actively produce the suspension having the desired density conveniently while preventing loss of the particles.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A density adjusting apparatus comprising:
a flow path structure including a main flow path into which a particle suspension flows and at least one branch flow path that branches from the main flow path and is provided with a concentration valve related to concentrating the particle suspension; and
processing circuitry configured to obtain density information indicating a density of particles contained in the particle suspension and goal information indicating a goal density of the particles contained in output liquid caused to flow out of the main flow path and configured to control opening and closing of the concentration valve on a basis of the density information and the goal information.

2. The density adjusting apparatus according to claim 1, wherein, to control the opening and closing of the concentration valve, the processing circuitry is configured to control one of: an opening or closing degree of the concentration valve; and fully opening/closing the concentration valve.

3. The density adjusting apparatus according to claim 1, further comprising:
a pump that is provided on an upstream side of the main flow path and is configured to cause the particle suspension to flow into the main flow path;
a transit liquid collecting tank that is provided on a downstream side of the branch flow path and is configured to collect transit liquid having transited through the branch flow path; and
an output liquid collecting tank that is provided on a downstream side of the main flow path and is configured to collect the output liquid having the goal density.

4. The density adjusting apparatus according to claim 3, wherein
the flow path structure further includes a merging flow path that merges with the main flow path on a downstream side of a most downstream branch point among branch points between the main flow path and the branch flow path and that is provided with a dilution valve for diluting the particle suspension on the downstream side,
the output liquid collecting tank is provided on a downstream side of a merging point between the merging flow path and the main flow path, and
processing circuitry is configured to control opening and closing of the dilution valve, on the basis of the density information and the goal information.

5. The density adjusting apparatus according to claim 4, further comprising: a diluted particle suspension input unit that is provided on an upstream side of the merging flow path and is configured to cause diluted liquid having a lower density than the particle suspension to flow into the merging flow path.

6. The density adjusting apparatus according to claim 1, further comprising:
a sensor configured to measure a density of the particles on an upstream side of a most upstream branch point among branch points between the main flow path and the branch flow path; and
an input interface configured to receive an input of the goal information.

7. The density adjusting apparatus according to claim 1, wherein the concentration valve is structured with a material and a mechanism that enable opening and closing thereof, according to a control signal output from the processing circuitry.

8. The density adjusting apparatus according to claim 7, wherein the material is a thermoresponsive polymer.

9. The density adjusting apparatus according to claim 8, wherein
a thin film electrode is attached to the thermoresponsive polymer, as the mechanism, and
the processing circuitry is configured to apply voltage to the thin film electrode, as the control signal.

10. The density adjusting apparatus according to claim 7, wherein
the material is an elastic material,
the mechanism is a mechanism configured to mechanically distort the elastic material, and
the processing circuitry is configured to output a signal for distorting the elastic material to the mechanism, as the control signal.

11. The density adjusting apparatus according to claim 1, wherein
the flow path structure further includes:
an extra main flow path different from the main flow path;
at least one extra branch flow path that branches from the extra main flow path and is provided with an extra concentration valve; and
a merging part where the main flow path merges with the extra main flow path, on a downstream side of a most downstream branch point among branch points between the main flow path and the branch flow path, and
the processing circuitry is configured to obtain extra density information indicating a density of other particles contained in another liquid caused to flow into the extra main flow path and extra goal information indicating a goal density of the other particles contained in another output liquid caused to flow out of the extra main flow path, and
the processing circuitry is configured to control opening and closing of the extra concentration valve, on a basis of the extra density information and the extra goal information.

12. The density adjusting apparatus according to claim 1, wherein
the processing circuitry is configured to further obtain output density information indicating a density of the particles contained in the output liquid, and
the processing circuitry is configured to further control opening and closing of the concentration valve on a basis of the output density information and the goal information.

13. A density-adjusted liquid producing method comprising:
causing a particle suspension to flow into a main flow path in a flow path structure including the main flow path into which the particle suspension flows and at least one branch flow path that branches from the main flow path and is provided with a concentration valve related to concentrating the particle suspension;
obtaining density information indicating a density of particles contained in the particle suspension and goal information indicating a goal density of the particles contained in output liquid caused to flow out of the main flow path; and
producing the output liquid having the goal density from the particle suspension, by controlling opening and closing of the concentration valve on a basis of the density information and the goal information.

14. The density-adjusted liquid producing method according to claim 13, wherein controlling the opening and closing of the concentration valve is to control one of: an opening or closing degree of the concentration valve; and fully opening/closing the concentration valve.

15. The density-adjusted liquid producing method according to claim 13, comprising:
causing the particle suspension to flow into the main flow path on an upstream side of the main flow path;
collecting transit liquid having transited through the branch flow path on a downstream side of the branch flow path; and
collecting the output liquid having the goal density on a downstream side of the main flow path.

16. The density-adjusted liquid producing method according to claim 15, wherein
the flow path structure further includes a merging flow path that merges with the main flow path on a downstream side of a most downstream branch point among branch points between the main flow path and the branch flow path and that is provided with a dilution valve for diluting the particle suspension on the downstream side,
an output liquid collecting tank for collecting the output liquid is provided on a downstream side of a merging point between the merging flow path and the main flow path, and
the density-adjusted liquid producing method comprises controlling opening and closing of the dilution valve on the basis of the density information and the goal information.

17. The density-adjusted liquid producing method according to claim 13, further comprising:
measuring a density of the particles on an upstream side of a most upstream branch point among branch points between the main flow path and the branch flow path; and
receiving an input of the goal information.

18. The density-adjusted liquid producing method according to claim 13, wherein the concentration valve is structured with a material and a mechanism that enable opening and closing thereof, according to a control signal based on the density information and the goal information.

19. The density-adjusted liquid producing method according to claim 13, wherein
the flow path structure further includes:
an extra main flow path different from the main flow path;
at least one extra branch flow path that branches from the extra main flow path and is provided with an extra concentration valve; and
a merging part where the main flow path merges with the extra main flow path, on a downstream side of a most downstream branch point among branch points between the main flow path and the branch flow path, and
the density-adjusted liquid producing method comprises:
obtaining extra density information indicating a density of other particles contained in another liquid caused to flow into the extra main flow path and extra goal information indicating a goal density of the other particles contained in another output liquid caused to flow out of the extra main flow path; and
controlling opening and closing of the extra concentration valve, on the basis of the extra density information and the extra goal information.

20. The density-adjusted liquid producing method according to claim 13, comprising:
further obtaining output density information indicating a density of the particles contained in the output liquid; and
further controlling opening and closing of the concentration valve on a basis of the output density information and the goal information.
